(19) United States
(12) Patent Application Publication
(10) Pub. No.: US 2007/0248677 A1
(43) Pub. Date: Oct. 25, 2007

(54) Method for treating warm-blooded vertebrates with a salt of a halide-free glucosamine base and a therapeutic drug

(75) Inventors: Alan Cowan, Ambler, PA (US); Robert B. Raffa, Norristown, PA (US); Ronald J. Tallarida, Mantua, NJ (US)

Correspondence Address: Law Offices of Jack Matalon, 32 Shelley Rd., Springfield, NJ, 07081-2529, US

(73) Assignee: Jame Fine Chemicals, Inc.

(21) Appl. No.: 11/223,295

(22) Filed: Sep. 9, 2005

Related U.S. Application Data
(60) Provisional application No. 60/611,211, filed on Sep. 17, 2004.

(51) Int. Cl. A61K 31/7008; A61K 9/14

(52) U.S. Cl. 424/487; 514/62

(57) ABSTRACT

A method of treating a warm-blood vertebrate. The verebrate may be a human being or a lower animal. The treatment method involves administering to the vertebrate in need of such treatment a pharmaceutically effective amount of a salt of a halide-free glucosamine base and a therapeutic drug containing at least one acid functionality, e.g., a carbonyl moiety, a carboxyl moiety, a sulfoxide moiety, etc. Preferably, the salt is stabilized by coating it with at least one pharmaceutically acceptable polymer comprising a water-soluble, water-immiscible and/or water-swellable homopolymer and/or copolymer. Suitable polymers include carboxypolymethylene homopolymers and copolymers; polyethylene glycol homopolymers and copolymers, povidone homopolymers and copolymers; polyacrylic acid homopolymers and copolymers; polyacrylamide homopolymers and copolymers; polysaccharides; and mixtures of two or more of the foregoing polymers. The resultant coated halide-free glucosamine-therapeutic drug salt composition will be stable upon exposure to ambient temperature and/or the atmosphere. Suitable therapeutic drugs containing at least one acid functionality may be found in one or more of the following classes of therapeutic drugs. α- and β-Adrenergic Agonists; Narcotic and Non-Narcotic Analgesics; Anorexics; Antiallergics; Antianginals; Antiarrhythmics; Antiasthmatics; Antibiotics; Anti-coagulants; Anticonvulsants; Antidepressants; Antidiabetics; Antihistaminics; Anti-hypertensives; Nonsteroidal Anti-Inflammatories; Antimigraines; Antineoplastics; Antiparkinsonians; Antipsychotics; Antipyretics; Antispasmodics; Antithrombotics; Anti-ulceratives; Anxiolytics; Decongestants; Diuretics; Hepatoprotectants; Sedatives; and Vasodilators.

### CROSS-REFERENCE TO RELATED APPLICATION

This application claims the benefit of provisional application Ser. No. 60/611,211 filed Sep. 17, 2004.

### FIELD OF THE INVENTION

The invention relates to a method for treating warm-blooded vertebrates, i.e., human beings and lower animals, with a salt of a halide-fee glucosamine base and a therapeutic drug containing at least one acidic functionality, i.e., one or more acidic moieties, e.g., a carbonyl group, a carboxyl group, a sulfoxide group, etc.

### BACKGROUND OF THE INVENTION

Glucosamine is a well-known amino monosaccharide found in chitin, glycoproteins and glycosaminoglycans. Glucosamine is widely used for the treatment of rheumatic fever, arthritic and arthosic complaints, in the acute as well as chronic forms, as well as in the treatment of pathological conditions originating from metabolic disorders of the osteo-articular tissue. Although products in the marketplace are labeled as, or referred to as, “glucosamine”, they are misnomers since such products consist of glucosamine hydrochloride or as unreacted mixtures of glucosamine hydrochloride and a salt such as potassium or sodium sulfate.

One drawback of many therapeutic drugs is their relative insolubility in the body after they have been administered to a patient. It would be most desirable if more soluble versions of therapeutic drugs could be made available.

It has now been found that salts of a halide-free glucosamine base and therapeutic drugs having an acidic functionality are more soluble than the therapeutic drugs themselves. An added benefit is that glucosamine itself is formed in the body (typically in the form of glucosamine phosphate) and therefore no “foreign” ingredients will be introduced in the body when the salt compositions employed in the treatment method of the invention are administered to a warm-blooded vertebrate.

Salts or mixtures of “glucosamine” or “glucosamine sulfate” and a therapeutic drug such as aspirin, ibuprofen, ketoprofen are known in the prior art, e.g., see U.S. Patent Publication 2002/0058642 A1; U.S. Pat. No. 6,608,041 B2; U.S. Pat. No. 6,291,527 B1; U.S. Pat. No. 5,604,206; and U.S. Pat. No. 3,008,874. However, the “glucosamine” or “glucosamine sulfide” employed in such compositions are misnomers, inasmuch as such materials are actually glucosamine hydrochloride or my salts of glucosamine hydrochoride and an alkali or alkaline earth metal sulfate. In contradistinction thereto, the glucosamine base employed in preparing the salt compositions employed in the method of the invention is halide free and as a result, the salt compositions will contain neither a halide nor any extraneous sulfate salts nor any extraneous cations (e.g., sodium potassium, calcium, etc.).

### DETAILS OF THE INVENTION

The invention pertains to a method for treating warm-blooded vertebrates. The method involves the administration to the vertebrate of a pharmaceutically effective amount of a salt of a halide-free glucosamine base and an acid therapeutic drug, i.e., a drug that exhibits a pH of lower than 7.0 because the drug contains one or more acidic functionalities such as a carbonyl moiety, a carboxyl moiety, a sulfoxide moiety, etc.

Descriptions of such salt compositions and the methods of preparation thereof appear in patent application Ser. No. ______, filed ______ (corresponding to provisional application Ser. No. 60/611,178 filed Sep. 17, 2004) filed on even date in the names of Vilas M. Chopdekar and Michael J. Torntore as the inventors. The disclosure of the foregoing patent application is incorporated herein in its entirety by reference and is summarized as set forth hereinbelow.

The starting materials for preparing the salts of a halide-free glucosamine-therapeutic drug employed in the treatment method of the invention are a halide-free glucosamine base and a therapeutic drug having at least one acid functionality, e.g., a carbonyl moiety, a carboxyl moiety and/or a sulfoxide moiety. Glucosamine, extracted from shellfish or prepared by a fermentation process, is only available in the form of its hydrochloride salt. If the glucosamine hydrochloride salt is neutralized with a base, e.g., NaOH, KOH, etc. in order to release the glucosamine base, the resultant product will always contain a salt, i.e., NaCl or KCl, respectively, and it is not possible to separate the glucosamine base from the salt since both the glucosamine base and the salt are fully soluble in water.

Free glucosamine base may be prepared by the method recited in Chem. Ber., volume 75, page 1274. Such method involves the treatment of glucosamine hydrochloride with an ethanolic solution of a tertiary base such as triethylamine. Triethylamine hydrochloride is filtered off and the free glucosamine is then recovered from the reaction mixture. However, triethylamine is a toxic material even in small quantities and the yield of the free glucosamine base is quite low. Moreover, the free glucosamine base still contains residual chloride.

The method for producing halide-free glucosamine base with a very high degree of purity may be summarized as follows:

(a) a glucosamine halide salt (e.g., glucosamine hydrochloride, glucosamine hydroiodide, etc.) is reacted with a lithium base in the presence of a C₁-C₄ alcohol to thereby generate a C₁-C₄ alcohol solution of a lithium halide and an insoluble halide-free glucosamine base; and

(b) the insoluble halide-free glucosamine base is separated from the C₁-C₄ alcohol solution of the lithium halide salt.

For maximum yields, the reaction should be carried out at a temperature of about 15 to about 35° C.; conveniently, the reaction may be carried out at ambient temperatures.

The C₁-C₄ alcohol may be, e.g., methanol, ethanol (preferably anhydrous), isopropanol, etc.; the preferred alcohol comprises methanol. The lithium base may be anhydrous lithium hydroxide, lithium hydroxide monohydrate, lithium methoxide, lithium ethoxide or lithium isopropoxide. The preferred lithium base comprises anhydrous lithium hydroxide. It has been found that the presence of water in the reaction mixture reduces the yield of the halide-free glucosamine base. Accordingly, it is preferred that the reaction be carried out under anhydrous conditions. In general, the lithium base is employed in an amount of about 1.0 to about 1.2 moles per mole of halide present in the glucosamine halide salt. Excess lithium base is unnecessarily wasteful and will reduce the yield of the halide-free glucosamine base. Typically, the alcohol is employed in an amount of about 1 to about 10 parts, preferably 3 to 6 parts, per part of lithium base.

After allowing the reaction to proceed (preferably with stirring) for about 5 minutes to about 2 hours, the solid halide-free glucosamine base is filtered off from the resultant alcohol solution of the lithium halide and washed with additional alcohol. The halide-free glucosamine base may then be dried under vacuum at a temperature of about 15 to about 30° C. The yield typically ranges from about 85 to about 90%. The halide-free glucosamine base is quite pure. It will have a purity level of greater than about 99 wt.% and the maximum halide content will be 0.01 wt.%, e.g., 100 ppm or less and very often, the halide content will be less than 50 ppm and as low as 25 ppm. Based upon the residual halide content of the halide-free glucosamine base, the lithium residue in the glucosamine base will generally be 20 ppm or less and very often, the lithium residue content will be less than 10 ppm.

The halide-free glucosamine base is quite hygroscopic and will decompose over a period of time if subjected to ambient temperature and/or to the atmosphere. Accordingly, it should be refrigerated in a closed container or preferably promptly used after recovery for conversion to the halide-free glucosamine-therapeutic drug salt of the invention by the method described below.

The halide-free glucosamine base may be readily converted to the halide-free glucosamine base-therapeutic drug salt employed in the treatment method of the invention by reacting it with a stoichiometric amount of a therapeutic drug having at least one acidic functionality. If the selected therapeutic drug has more than one acidic functionality, the molar ratio of the halide-free glucosamine base to the selected drug should be adjusted that there will be one mole of the glucosamine base employed per acidic functionality in the selected drug. Typically, the reaction mixture will comprise the halide-free glucosamine base, about 5 to about 30 parts, preferably 15 to 20 parts, of water (preferably purified water) per part of the base and the selected drug. Although lesser amounts of water may be employed, the resultant solutions may become too viscous to be properly agitated, particularly if the halide-free glucosamine-therapeutic drug salt composition is not isolated from the reaction mixture, but is stabilized by the addition of a polymer to the reaction mixture, as described below. On the other hand, excessive amounts of water may lead to reduced yields if a water-miscible solvent is used to recover the composition and if freeze-drying is used to recover the composition, the freeze-drying process becomes more time-consuming and expensive because of the large amount of water to be removed from the reaction mixture.

The selected therapeutic drug is slowly added to the aqueous solution of the halide-free glucosamine base while the aqueous solution is agitated, e.g. over a period of a few minutes, and the reaction mixture is further agitated for 5 to 120 minutes. The reaction is typically conducted at a temperature of about 15 to about 40° C.

Thereafter, the glucosamine base-therapeutic drug salt employed in the treatment method of the invention may be recovered from the reaction mixture by freeze-drying or by adding a water-miscible solvent such as acetone to the reaction mixture such that the composition will precipitate from the reaction mixture and the glucosamine base-therapeutic drug salt is then recovered by conventional filtration methods. The glucosamine base-therapeutic drug salt composition may then be dried by conventional methods, e.g., a stream of nitrogen, a vacuum oven at 30-50° C. for a period of 1 to 10 hours, etc. It is preferred that the recovery of the glucosamine base-therapeutic drug salts be carried out by a freeze-drying process as described in greater detail below.

Some of the glucosamine base-therapeutic drug salts employed in the treatment method of the invention may decompose over a period of time if they are exposed to ambient temperatures or the atmosphere. Therefore, it is preferred that the glucosamine base-therapeutic drug salt not be recovered from the reaction mixture as is, but converted to a stabilized form prior to recovery. Conversion of the salt to its stabilized form may be desirable even for those salts that do not decompose upon exposure to ambient temperatures and/or the atmosphere, since the pharmaceutically acceptable polymers employed in stabilizing, i.e., coating, the salt may provide extended-release properties when the salt is administered to warm-blooded vertebrates in accordance with the invention.

Stabilization of the glucosamine base-therapeutic drug salt is readily accomplished by adding a suitable pharmaceutically acceptable polymer to the reaction mixture prior to recovery of the composition. The pharmaceutically acceptable polymer may be a water-soluble, water-dispersible and/or or a water-swellable homopolymer and/or copolymer. Preferably, the pharmaceutically acceptable polymer will be water-soluble. In general, the polymer will be employed in an amount of about 2 to about 70, preferably 20 to 50, parts by weight of the polymer per part of the composition in the reaction mixture.

Nonlimiting examples of commercially available pharmaceutically acceptable homopolymers and copolymers suitable for stabilizing the halide-free glucosamine-drug composition employed in the treatment method of the invention include the following: carboxypolymethylene homopolymers and copolymers, i.e., vinyl polymers having active carboxyl groups such as high molecular weight homopolymers of acrylic acid crosslinked with allylsucrose or allylpentaerythritol and copolymers of acrylic acid modified by long chain (C₁₀-C₃₀) alkyl acrylates and crosslinked with allylpentaerythritol—such polymers are commercially available and are marketed as Carbopol® polymers; polyethylene glycol homopolymers, particularly polyethylene glycol homopolymers having molecular weights in the range of about 4,000 to about 8,000; polyethylene glycol copolymers such as polyethylene-co-lactic acid copolymers; polypropylene glycol homopolymers and copolymers, especially polypropylene glycol homopolymers having molecular weights of about 800 to about 3,000; povidone homopolymers, i.e., synthetic water-soluble homopolymers of N-vinyl-pyrrolidone, especially those having a molecular weight of about 2,500 to about 10,000; copovidone, i.e. synthetic random copolymers of N-vinylpyrrolidone and vinyl acetate in a 60:40 ratio; polyacrylic acid homopolymers and copolymers; polyacrylamide homopolymers and copolymers; polysaccharides; etc.

The choice of particular homopolymers and/or copolymers for coating, i.e., stabilizing, the halide-free glucosamine-therapeutic drug salt composition, is not critical so long as the polymers are pharmaceutically acceptable, have the capability of coating, i.e., stabilizing, the halide-free glucosamine-therapeutic drug salt composition without any adverse chemical reaction occurring between the selected polymer and the halide-free glucosamine-therapeutic drug salt and the resultant coated glucosamine base-therapeutic drug salt compositions are stable, i.e., they will not undergo decomposition when exposed to ambient temperatures and/or the atmosphere.

If the glucosamine base-therapeutic drug salt is to be recovered from the reaction mixture in a stabilized form, the desired pharmaceutically acceptable polymer is added, preferably in increments, with stirring, to the aqueous glucosamine base solution preferably prior to the addition of the therapeutic drug. This step will generally take about 5 to about 15 minutes and is preferably conducted at a temperature of about 15 to about 40° C. After all increments of the selected polymer have been added, stirring is continued for an additional 5 to 120 minutes. Thereafter, the organic acid is slowly added to the reaction mixture, while maintaining the reaction mixture at a temperature of about 15 to about 40° C.

The last step is the recovery of the polymer-coated, i.e., stabilized, glucosamine base-therapeutic drug salt from the reaction mixture. The stabilized glucosamine base-therapeutic drug salt composition may be recovered from the reaction mixture by freeze-drying or by adding a water-miscible solvent, e g., acetone, to the reaction mixture to cause the stabilized glucosamine-therapeutic drug salt composition to precipitate out from the reaction mixture. The precipitate is then recovered by conventional filtration methods and it may be dried as described below. Of course, the choice of stabilizing polymer and water-miscible solvent should be such that the polymer will not dissolve in, or otherwise react with, the solvent.

The stabilized glucosamine base-therapeutic drug salt composition is preferably recovered by removal of water from the reaction mixture by a well-known technique for removing water from compositions. Although freeze-drying is a time-consuming process, (a reaction mixture containing one liter of water will typically require 30-36 hours to remove about 97% of the water), it is preferred since the formation of decomposition products resulting from heating the reaction mixture or adding solvents to the reaction mixture can be avoided.

The freeze-drying process will generally be carried out at a reduced pressure and reduced temperature, e.g., a pressure of not greater than 500 milliTorre, preferably 300 to 100 milliTorre and at a temperature of about 60 to about −20° C., preferably −50 to −40° C. The endpoint of the completion of the freeze-dying process may be determined by condensing and measuring the quantity of water removed during the freeze-drying process. The time required for completion of the freeze-drying process will vary depending on factors such as pressure, temperature, quantity of reaction mixture to be free-dried, level of water to be tolerated in the stabilized halide-free glucosamine-drug composition, the thickness and surface area of the reaction mixture in the trays of the freeze-drying equipment, etc.

If the stabilized halide-free glucosamine-therapeutic drug salt composition is to be recovered by precipitation from the reaction mixture by addition of a water-miscible solvent such as acetone to the reaction mixture, generally about 2 to about 10 parts of solvent per part of reaction mixture will be required.

After the stabilized glucosamine-therapeutic drug salt composition has been recovered from the reaction mixture, it may be dried by conventional techniques, e.g., a stream of nitrogen, vacuum oven at a temperature of about 30 to about 50° C. for 1 to 10 hours or more, etc.

It should also be noted that the stabilization of the halide-free glucosamine-therapeutic drug salt compositions employed in the treatment method of the invention may provide an additional advantage to warm-blooded vertebrates to whom such compositions are administered. The stabilized, i.e., polymer-coated, versions of the glucosamine base-drug salts may provide extended release properties, i.e., the glucosamine base-therapeutic drug salt may be released the vertebrate over an extended period of time, thereby possibly resulting in a reduction of the frequency and the amount of the dosage that would otherwise be required to be administered to the vertebrate.

The therapeutic drug that is to be saltified with the halide-free glucosamine base may be any therapeutic drug that exhibits an acidic pH, i.e., a pH of lower than 7.0. Such drugs will contain one or more acidic functionalities such as a carbonyl-moiety, a carboxyl moiety, a sulfoxide moiety, etc. The list of therapeutic drugs that fit such definition is quite voluminous.

Suitable therapeutic drugs containing at least one acid functionality may be found in one or more of the following representative classes of drugs: α- and β-Adrenergic Agonists; Narcotic and Non-Narcotic Analgesics; Anorexics; Antiallergics; Antianginals; Antiarrhythmics; Antiasthmatics; Antibiotics; Antibiotics, Anticoagulants; Anti-convulsants; Antidepressants; Antidiabetics; Antihistaminics; Antihypertensives; Nonsteroidal Anti-inflammatories; Antimigraines; Antineoplastics; Anti-parkinsonians; Antipsychotics; Antipyretics; Antispasmodics; Antithrombotics, Antiulceratives; Anxiolytics; Decongestants; Diuretics, Hepatoprotectants; Sedatives; Vasodilators; and the like.

Not every possible therapeutic drug within the foregoing-listed classes will be suitable for preparing a salt with the glucosamine base. Only those therapeutic-drugs that are sufficiently acidic in nature to form such a salt with the halide-free glucosamine base are suitable. As mentioned above, such therapeutic drugs will have a pH of lower than 7.0 and will contain at least one acid functionality e.g. a carbonyl moiety, a carboxyl moiety, a sulfoxide, moiety, etc.

Particularly suitable specific drugs within the foregoing classes include: acetaminophen, acetazolamide, ampicillin, ampiroxicam, aspirin, bromfenac, celecoxib, cetirizine, chlorothiazide, chlorpropamide, ciprofloxacin, diclofenac, ethacrynic acid, flufenamic acid, furosemide, ibuprofen, indomethacin, indoprofen, ketoprofen, levodopa, meclofenamic acid, methotrexate, methyldopa, naproxen, orazamide, penicillamine, pentobarbital, phenobarbital, phenytoin, piroxicam, propylthiouracil, protoprophyrin IX, rofecoxib, salicyclic, acid, sulfadiazine, sulfapyridine, sulindac, theophylline, thioctic acid, timonacic, tiopronin, tolbutanide, tolfenamic acid, warfarin, tolmetin, zaltoprofen, and the like.

The treatment method of the invention involves the administration of a pharmaceutically effective amount of one or more of the foregoing salts to a warm-blooded vertebrate in need of such treatment. It should be understood that the term “warm-blooded vertebrate” is intended to encompass human beings as well as lower animals, e.g., dogs, cats, horses, cattle, swine, poultry, etc.

The pharmaceutically effective amount of the salt of the glucosamine base and an acidic therapeutic drug may be administered to the warm-blooded vertebrate in need of such treatment by one or more of a variety of conventional methods, e.g., orally, buccally, intravenously, intramuscularly, parenterally, sublingually, topically, etc. The salt compositions employed in the treat invention may be utilized in the form of tablets, caplets, granules, powder, capsules, spansules, gel caps, solutions, syspensions, syrups, mouthwashes, salves, foams, gels, creams, vaginal bougies, suppositories, and the like.

If desired, the salts administered in accordance with the treatment method of the invention may also contain pharmaceutically effective amounts of other therapeutic drugs that would not form salts with the glucosamine base. Nonlimiting examples of such other therapeutic drugs include albuterol, allopurinol, alprenolol, amiloride, amiodarone, amphetamine, atropine, bupivacaine, chlordiazepoxide, chloroquine, chlorpheniramine, chlorpromazine, clonidine, cocaine, codeine, cyclizine, desipramine, diazepam, dihydrocodeine, diphenhydramine, diphenoxylate, ephedrine, epinephrine, ergotamine, fluphenazine, guanethidine, hydralazine, imipramine, isoproterenol, kanamycin, lidocaine, metaraminol, methadone, methamphetamine, metoprolol, morphine, nicotine, norepinephrine, oxymetazoline, pentazocine, phenylephrine, physostigmine, pilocarpine, pindolol, procainamide, procaine, promazine, promethazine, propranolol, pseudoephedrine, pyrimethamine, quinidine, scopalamine, strychnine, terbutaline, thioridazine, tolazoline, and the like.

The following nonlimiting examples shall serve to illustrate the preferred embodiments of the invention. Unless otherwise indicated, all parts and percentages are on a weight basis.

### EXAMPLE 1

The halide-free glucosamine salts employed in the method of the invention were supplied by Jame Fine Chemicals, Inc., the assignee of patent application Ser. No. ______, filed ______ (corresponding to provisional application Ser. No. 60/611,178 filed Sep. 17, 2004) filed on even date in the names of Vilas M. Chopdekar and Michael J. Torntore as the inventors. The glucosamine salts were prepared in accordance with the teachings of the aforesaid co-ending application that is incorporated herein in its entirety by reference. The test that was employed is an abdominal irritant test that was carried out in accordance with the procedure set forth by Collier et al., Brit. J. Pharmacol., 32:295-310 (1968).

Each group of 10 male Swiss-Webster mice weighing 25-30 g received an oral injection of (a) distilled water or 1% Tween® 80; or (b) a previously established analgesic dose of either ibuprofen (30 mg/kg), ketoprofen (200 mg/kg), naproxen (30 mg/kg), piroxicam (3 mg/kg) or aspirin (150 mg/kg) or (c) a halide-free glucosamine salt of either ibuprofen (30 mg/kg of the salt), ketoprofen (200 mg/kg of the salt), naproxen (30 mg/kg of the salt), piroxicam (3 mg/kg of the salt) or aspirin (150 mg/kg of the salt).

It was noted that the halide-free glucosamine salts of ibuprofen, ketoprofen, naproxen, piroxicam and aspirin were all 100% soluble in distilled water in contrast to the parent compounds which had to be suspended in water containing 1% Tween® 80.

Thirty minutes after each injection, each mouse was injected i.p. (0.25 ml/0.25 g) with an aqueous solution of acetylcholine bromide (5.5 mg/kg). The animals were then observed for 10 minutes for the presence or absence of a characteristic wave of contraction and elongation passing caudually along the abdominal wall, accompanied by a twisting of the trunk and followed by extension of the hind limbs. The percentage of analgesia was then calculated for each group based on the percentage of mice displaying no behavioral response.

The results of the abdominal irritant test were as follows:

At 30 mg/kg, both ibuprofen and the halide-free glucosamine salt of ibuprofen (containing 53% ibuprofen) provided 80% analgesia.

At 200 mg/kg, ketoprofen and the halide-free glucosamine salt of ketoprofen (containing 59% ketoprofen) provided 56% and 90% analgesia, respectively.

At 30 mg/kg, naproxen and the halide-free glucosamine salt of naproxen (containing 46% naproxen) provided 70% and 50% analgesia, respectively.

At 3 mg/kg, both piroxicam and the halide-free glucosamine salt of piroxicam (containing 65% piroxicam) provided 70% analgesia.

At 50 mg/kg, both aspirin and the halide-free glucosamine salt of aspirin (containing 50% aspirin) provided 70% analgesia.

Based on the above result, it is clear that the halide-free glucosamine salt of each non-steroidal anti-inflammatory drug was a more potent analgesic on a proportional content basis than the parent drug.

What is claimed is:

1. A method of treating a warm-blooded vertebrate comprising the steps of:
(a) providing a salt of a glucosamine base having a purity level of at least about 99 wt.% and a maximum halide content of 0.01 wt.%, and a therapeutic drug having at least one acid functionalityl; and
(b) administering to the vertebrate in need of the treatment a pharmaceutically effective amount of such salt.

2. The method of claim 1 wherein the salt further comprises a pharmaceutically acceptable polymer.

3. The method of claim 4 wherein the polymer comprises a water-soluble, water-dispersible and/or a water-swellable homopolymer and/or copolymer.

4. The method of claim 2 wherein, the polymer is selected from the group consisting of carboxypolymethylene homopolymers and copolymers; polyethylene glycol homopolymers and copolymers, povidone homopolymers and copolymers; polyacrytic acid homopolymers and copolymers; polyactylamide homopolymers and copolymers; polysaccharides; and mixtures of two or more of the foregoing polymers.

5. The method of claim 1 wherein the therapeutic drug is selected from the group consisting of the classes of α- and β-Adrenergic Agonists; Narcotic and Non-Narcotic Analgesics; Anorexics; Antiacne and Keratolytics; Antiallergics; Antianginals; Antiarrhythmics; Antiasthmatics; Antibiotics; Anticoagulants; Anticonvulsants; Antidepressants; Antidiabetics; Antihistaminics; Antihypertensives; Nonsteroidal Anti-Inflammatories; Antimigraines Antineoplastics; Antiparkinsonians; Antipsychoticis; Antipyretics; Antispasmodics; Antithrombotics; Antiulceratives; Anxiolytics; Diuretics; Decongestant; Hepatprotectants; Sedatives; and Vasodilators.

6. The method of claim 5 wherein the drug is selected from the group consisting of acetaminophen, acetazolamide, ampicillin, ampiroxicam, aspirin, bromfenac, celecoxib, chlorothiazide, chlorpropamide, cetirizine, ciprofloxacin, diclofenac, ethacrynic acid, flufenamic acid, furosemide, ibuprofen, indomethacin, indoprofen, ketoprofen, levodopa, meclofenamic acid, methotrexate, methyldopa, naproxen, orazamide, penicillamine, pentobarbital, phenobarbital, phenytoin, piroxicam, propylthiouracil, protoprophyrin IX, rofecoxib, salicyclic acid, sulfadiazine, sulfapyridine, sulindac, theophylline, thioctic acid, timonacic, tiopronin, tolbutamide, tolfenamic acid, warfarin, tolmetin, zaltoprofen, and mixtures thereof.

7. The method of claim 1 wherein the salt is administered to the vertebrate in conjunction with, and/or in admixture with, with a pharmaceutically effective amount of one or more other therapeutic drugs that would not form a salt with the glucosamine base.

8. The method of claim 7 wherein such other therapeutic drugs are selected from the group consisting of albuterol, allopurinol, alprenolol, amiloride, amiodarone, amphetamine, atropine, bupivacaine, chlordiazepoxide, chloroquine, chlorpheniramine, chlorpromazine, clonidine, cocaine, codeine, cyclizine, desipramine, diazepam, dihydrocodeine, diphenhydramine, diphenoxylate, ephedrine, epinephrine, ergotamine, fluphenazine, guanethidine, hydralazine, imipramine, isoproterenol, kanamycin, lidocaine, metaraminol, methadone, methamphetamine, metoprolol, morphine, nicotine, norepinephrine, oxymetazoline, pentazocine, phenylephrine, physostigmine, pilocarpine, pindolol, procainamide, procaine, promazine, promethazine, propranolol, pseudoephedrine, pyrimethamine, quinidine, scopalamine, strychnine, terbutaline, thioridazine, tolazoline and mixtures thereof.

9. The method of claim 1 wherein the salt is administered to the vertebrate orally, buccally, intravenously, intramuscularly, parenterally, sublingually and/or topically.

10. The method of claim 1 wherein the salt is utilized in the form of tablets, caplets, granules, powder, capsules, spansules, gel caps, solutions, suspensions, syrups, mouthwashes, salves, foams, gels, creams, vaginal bougies and/or suppositories.

11. A method of treating a warm-blooded vertebrate comprising the steps of:
(a) providing a pharmaceutically acceptable polymer-coated salt of a glucosamine base having a purity level of at least about 99 wt. % and a maximum halide content of about 0.01 wt. %, and a therapeutic drug containing at least one acid functionality; and
(b) administering a pharmaceutically effective amount of such polymer-coated salt to the verebrate in need of the treatment.

12. The method of claim 11 wherein the pharmaceutically acceptable polymer comprises a water-soluble, water-dispersible and/or a water-swellable homopolymer and/or copolymer.

13. The method of claim 11 wherein the pharmaceutically acceptable polymer is selected from the group consisting of carboxypolymethylene, homopolymers, and copolymers; polyethylene glycol homopolymers and copolymers, povidone homopolymers and copolymers; polyacrylic acid homopolymers and copolymers; polyacrylamide homopolymers and copolymers; polysaccharides; and mixtures of two or more of the foregoing polymers.

14. The method of claim 11 wherein the therapeutic drug is selected from the group consisting of the classes of α- and β-Adrenergic Agonists; Narcotic and Non-Narcotic Analgesics; Anorexics; Antiacne and Keratolytics; Antiallergics; Antianginals; Antiarrhythmics; Antiasthmatics; Antibiotics; Anticoagulants; Anticonvulsants; Antidepressants; Antidiabetics; Antihistaminics; Antihypertensives, Nonsteroidal Anti-Inflammatories; Antimigraines Antineoplastics; Antiparkinsonians; Antipsychotics; Antipyretics; Antispasmodics; Antithrombotics; Antiulceratives; Anxiolytics; Diuretics; Decongestants; Hepatoprotectants; Sedatives; and Vasodilators.

15. The method of claim 14 wherein the therapeutic drug is selected from the group consisting of acetaminophen, acetazolamide, ampicillin, ampiroxicam, aspirin, bromfenac, celecoxib, cetirizine; chlorothiazide, chlorpropamide, ciprofloxacin, diclofenac, ethacrynic acid, flufenamic acid, furosemide, ibuprofen, indomethacin, indoprofen, ketoprofen, levodopa, meclofenamic acid, methotrexate, methyldopa, naproxen, orazamide, penicillamine, pentobarbital, phenobarbital, phenytoin, piroxicam, propylthiouracil, protoprophyrin IX, rofecoxib, salicylic acid, sulfadiazine, sulfapyridine, sulindac, theophylline, thioctic acid, timonacic, tiopronin, tolbutamide, tolfenamic acid, warfarin, tolmetin, zaltoprofen, and mixtures thereof.

16. The method of claim 11 wherein the pharmaceutically acceptable polymer-coated salt is administered to the vertebrate orally, buccally, intravenously, intramuscularly, parenterally, sublingually and/or topically.

17. The method of claim 11 wherein the pharmaceutically acceptable polymer-coated salt is utilized in the form of tablets, caplets, granules, powder, capsules, spansules, gel caps, solutions, suspensions, syrups, mouthwashes, salves, foams, gels, creams, vaginal bougies and/or suppositories.

18. The method of claim 11 wherein the pharmaceutically acceptable polymer-coated salt is administered to the vertebrate in conjunction with, and/or in admixture with, with a pharmaceutically effective amount of one or more, other therapeutic drugs that would not form a salt with the glucosamine base.

19. The method of claim 11 wherein such other therapeutic drugs are selected from the group consisting of albuterol, allopurinol, alprenolol, amiloride, amiodarone, amphetamine, atropine, bupivacaine, chlordiazepoxide, chloroquine, chlorpheniramine, chlorpromazine, clonidine, cocaine, codeine, cyclizine, desipramine, diazepam, dihydrocodeine, diphenhydramine, diphenoxylate, ephedrine, epinephrine, ergotamine, fluphenazine, guanethidine, hydralazine, imipramine, isoproterenol, kanamycin, lidocaine, metaraminol, methadone, methamphetamine, metoprolol, morphine, nicotine, norepinephrine, oxymetazoline, pentazocine, phenylephrine, physostigmine, pilocarpine, pindolol, procainamide, procaine, promazine, promazine, propranolol, pseudoephedrine, pyrimethamine, quinidine, scopalamine, strychnine, terbutaline, thioridazine, tolazoline and mixtures thereof.

20. The method of claim 11 wherein the polymer is present in an amount of about 2 to about 70 parts by weight per part of the salt.
